(19) United States
(12) Patent Application Publication
(10) Pub. No.: US 2005/0014938 A1
(43) Pub. Date: Jan. 20, 2005

(54) Phenyl substituted triazoles and their use as selective inhibitors of akl5 kinase

(76) Inventors: Laramie Mary Gaster, Essex (GB); John David Harling, Hertfordshire (GB); Jag Paul Heer, Essex (GB); Thomas Daniel Heightman, Essex (GB); Andrew Hele Payne, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/495,414

(22) PCT Filed: Nov. 14, 2002
(86) PCT No.: PCT/EP02/13482

(30) Foreign Application Priority Data
Nov. 15, 2001 (GB) 0127430.7

(51) Int. Cl. C07D 417/02; C07D 413/02; C 07D 4 3/02

(52) U.S. Cl. 544/60; 544/139; 544/140; 544/370; 544/371; 546/268.4; 546/276.7; 546/210; 546/211

(57) ABSTRACT

Phenyl substituted triazoles of formula (I) 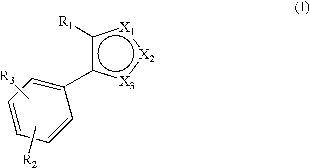
wherein R₁ is naphthyl or phenyl optionally substituted with one or more substituents selected from halo, —O—C₁₋₆alkyl, —S—C₁₋₆alkyl, C₁₋₆alkyl, C₁₋₆haloalkyl, —O—(CH₂)ₙ-Ph, —S—(CH₂)ₙ-Ph, cyano, phenyl, and CO₂R, wherein R is hydrogen or C₁₋₆alkyl, and n is 0, 1, 2 or 3; or R₁ is phenyl or pyridyl fused with an aromatic or non-aromatic cyclic ring of 5-7 members wherein said cyclic ring optionally contains up to three heteroatoms, independently selected from N, O and S, and N may be further optionally substituted by C₁₋₆ alkyl, and wherein the cyclic ring may be optionally substituted by ═O;
R₂ and R₃ are independently selected from H, C₁₋₆alkyl, C₁₋₆alkoxy, phenyl, NH(CH₂)ₙ-Ph, NH—C₁₋₆alkyl, halo, alkoxy, CN, NO₂, CONHR and SO₂NHR;
two of X₁, X₂ and X₃ are N and the other is NR₄ wherein R₄ is hydrogen, C₁₋₆alkyl, C₃₋₇cycloalkyl, —(CH₂)ₚ—CN, —(CH₂)ₚ—CO₂H, —(CH₂)ₚ—CONHR₅R₆, —(CH₂)ₚCOR₅, —(CH₂)_{q}(CR₇)₂, —(CH₂)ₚOR₅, (CH₂)_{q}CH═CH—CN, —(CH₂)_{q}—CH═CH—CO₂H, —(CH₂)ₚ—CH═CH—CONHR₅R₆, —(CH₂)ₚNHCOR₈ or —(CH₂)ₚNR₉R₁₀;
R₅ and R₆ are independently hydrogen or C₁₋₆alkyl;
R₇ is C₁₋₆alkyl;
R₈ is C₁₋₇alkyl, or optionally substituted aryl, heteroaryl, arylC₁₋₆alkyl or heteroaryl C₁₋₆alkyl;
R₉ and R₁₀ are independently selected from hydrogen, C₁₋₆alkyl, aryl and arylC₁₋₆alkyl; p is 0-4; and q is 1-4 and salts and solvates thereof, are disclosed, as are methods for their preparation, pharmaceutical compositions containing them and their use in medicine.

This invention relates to phenyl substituted triazoles which are inhibitors of the transforming growth factor, (“TGF”)-β signaling pathway, in particular, the phosphorylation of smad2 or smad3 by the type I or activin-like kinase (“ALK”)-5 receptor, methods for their preparation and their use in medicine.

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided in two classes, the type I or activin-like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signaling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. Generally it is believed that in many species, the type II receptors regulate cell proliferation and the type I receptors regulate matrix production. Therefore, preferred compounds of this invention are selective in that they inhibit the type I receptor and thus matrix production, and not the type II receptor mediated proliferation.

Activation of the TGF-β1 axis and expansion of extracellular matrix are early and persistent contributors to the development and progression of chronic renal disease and vascular disease. Border W. A., Noble N. A., N. Engl. J. Med., Nov. 10, 1994; 331(19):1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., Feng X. H., Derynck R., Nature, Aug. 27, 1998; 394(6696):909-13; Usui T., Takase M., Kaji Y., Suzuki K., Ishida K., Tsuru T., Miyata K., Kawabata M., Yamashita H., Invest. Ophthalmol. Vis. Sci., October 1998; 39(11):1981-9.

Progressive fibrosis in the kidney and cardiovascular system is a major cause of suffering and death and an important contributor to the cost of health care. TGF-β1 has been implicated in many renal fibrotic disorders. Border W. A., Noble N. A., N. Engl. J. Med., Nov. 10, 1994; 331(19):1286-92. TGF-β1 is elevated in acute and chronic glomerulonephritis, Yoshioka K., Takemura T., Murakami K., Okada M., Hino S., Miyamoto H., Maki S., Lab. Invest., February 1993; 68(2):154-63, diabetic nephropathy, Yamamoto, T., Nakamura, T., Noble, N. A., Ruoslahti, E., Border, W. A., (1993) PNAS 90:1814-1818, allograft rejection, HIV nephropathy and angiotensin-induced nephropathy, Border W. A., Noble N. A., N. Engl. J. Med., Nov. 10, 1994; 331(19):1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., Factor V. M., Mozes M., Nagy P., Sanderson N., Bottinger E. P., Klotman P. E., Thorgeirsson S. S., Lab Invest, June 1996; 74(6):991-1003. Thus, inhibition of TGF-β1 activity is indicated as a therapeutic intervention in chronic renal disease.

TGF-β1 and its receptors are increased in injured blood vessels and are indicated in neointima formation following balloon angioplasty, Saltis J., Agrotis A., Bobik A., Clin Exp Pharmacol Physiol, March 1996; 23(3):193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., Wang J. M., Wilson P. B., Kumar S., Atherosclerosis, February 1996; 120(1-2):221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., Consigli S., Du B., Falcone D. J., Sanborn T. A., Spokojny A. M., Bush H. L., Jr., J Clin Invest, December 1995; 96(6):2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodeling via a TGF-β-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1 signaling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signaling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

TGFβ1-antibodies prevent transplanted renal tumor growth in nude mice through what is thought to be an anti-angiogenic mechanism Ananth S. et al, Journal Of The American Society Of Nephrology Abstracts, 9: 433A(Abstract). While the tumor itself is not responsive to TGF-β, the surrounding tissue is responsive and supports tumor growth by neovascularization of the TGF-β secreting tumor. Thus, antagonism of the TGF-β pathway should prevent metastasis growth and reduce cancer burden.

Surprisingly, it has now been discovered that a class of phenyl substituted triazoles function as potent and selective non-peptide inhibitors of ALK5 kinase and therefore, have utility in the treatment and prevention of various disease states mediated by ALK5 kinase mechanisms, such as chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis and restenosis.

Examples of diseases where fibrosis is a major component include, but are not limited to, hepatitis B virus (HBV), hepatitis C virus (HCV), alcohol-induced hepatitis, haemochromatosis and primary biliary cirrhosis.

According to the invention there is provided a compound of formula (1) or a pharmaceutically acceptable salt thereof: 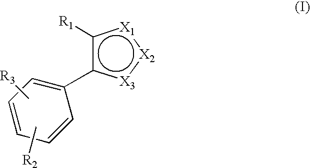
wherein R₁ is naphthyl or phenyl optionally substituted with one or more substituents selected from halo, —O—C₁₋₆alkyl, —S—C₁₋₆alkyl, C₁₋₆alkyl, C₁₋₆haloalkyl, —O—(CH₂)ₙ-Ph, —S—(CH₂)ₙ-Ph, cyano, phenyl, and CO₂R, wherein R is hydrogen or C₁₋₆alkyl, and n is 0, 1, 2 or 3; or R₁ is phenyl or pyridyl fused with an aromatic or non-aromatic cyclic ring of 5-7 members wherein said cyclic ring optionally contains up to three heteroatoms, independently selected from N, O and S, and N may be further optionally substituted by C₁₋₆ alkyl, and wherein the cyclic ring may be optionally substituted by ═O;
R₂ and R₃ are independently selected from H, C₁₋₆alkyl, C₁₋₆alkoxy, phenyl, NH(CH₂)ₙ-Ph, NH—C₁₋₆alkyl, halo, CN, NO₂, CONHR and SO₂NHR;
two of X₁, X₂ and X₃ are N and the other is NR₄ wherein R₄ is hydrogen, C₁₋₆alkyl, C₃₋₇cycloalkyl, —(CH₂)ₚ, —(CH₂)ₚ—CO₂H, —(CH₂)ₚ—CONHR₅R₆, —(CH₂)ₚCOR₅, —(CH₂)_{q}(OR₇)₂, —(CH₂)ₚOR₅, —(CH₂)_{q}—CH═CH—CN, —(CH₂)_{q}—CH═CO₂H, —(CH₂)ₚ—CH—CH—CONHR₅R₆, —(CH₂)ₚNHCOR₈ or —(CH₂)ₚNR₉R₁₀;
R₅ and R₆ are independently hydrogen or C₁₋₆alkyl;
R₇ is C₁₋₆alkyl;
R₈ is C₁₋₇alkyl, or optionally substituted aryl, heteroaryl, arylC₁₋₆alkyl or heteroarylC₁₋₆alkyl;
R₉ and R₁₀ are independently selected from hydrogen, C₁₋₆alkyl, aryl and arylC₁₋₆alkyl;
p is 0-4; and
q is 14.

In the triazole ring of the compounds of formula (I it will be apparent that the double bond will be to the two unsubstituted nitrogens.

When R₁ is pyridyl fused with an aromatic or non-aromatic cyclic ring of 5-7 members the nitrogen of the pyridyl ring may be at the point of fusion. Preferably R₁ is optionally substituted naphthyl or phenyl. More preferably R₁ is phenyl optionally substituted with one or more substituents selected from halo, C₁alkoxy, C₁alkylthio, and phenyl; or R₁ is phenyl or pyridyl fused with an aromatic or non-aromatic cyclic ring of 5-7 members wherein said cyclic ring optionally contains up to three heteroatoms, independently selected from N, O and S, and N may be further optionally substituted by C₁₋₆ alkyl, and wherein the cyclic ring may be optionally substituted by ═O; for example R₁ represents benzo[1,3]dioxolyl, 2,3-dihydrobenzo[1,4]dioxinyl, benzoxazolyl, benzothiazolyl, benzo[1,2,5]oxadiazolyl, benzo[1,2,5]thiadiazolyl, quinoxalinyl, dihydrobenzofuranyl, benzimidazolyl, C₁₋₆ alkylbenzimidazolyl, [1,2,4]triazolo[1,5-a]pyridyl, benzo[1,4]oxazinyl-3-one, benzoxazolyl-2-one or benzo[1,4]oxazinyl. Most preferably R₁ represents 4-methoxyphenyl, 3-fluoro-4-methoxyphenyl, 3-chlorophenyl, 3-fluoro-4-methoxyphenyl or 3-chloro-4-methoxyphenyl, or R₁ represents benzo[1,2,5]thiadiazolyl, [1,2,4]triazolo[1,5-a]pyridyl, dihydrobenzofuranyl, 2,3-dihydrobenzo[1,4]dioxinyl, benzimidazolyl, C₁₋₆ alkylbenzimidazolyl, benzo[1,4]oxazinyl-3-one or benzo[1,4]oxazinyl.

Preferably, R₂ is positioned meta to the point of attachment to the triazole, R₂ is preferably halo, e.g. chloro, C₁alkyl or NO₂. More preferably, R₂ is halo.

R₃ is preferably hydrogen or halo.

The compounds for use in the methods of the invention preferably have a molecular weight of less than 800, more preferably less than 600.

Specific compounds of the invention which may be mentioned include the following and pharmaceutically acceptable salts thereof:
6-[5-(3-Chlorophenyl)-1H-[1,2,3]triazol-4-yl]-[1,2,4]triazolo[1,5-a]pyridine;
6-[5-(3-Fluorophenyl)-1H-[1,2,3]triazol-4-yl]-[1,2,4]triazolo[1,5-a]pyridine;
6-[5-(3-Nitrophenyl)-1H-[1,2,3)triazol-4-yl]-[1,2,4]triazolo[1,5-a]pyridine;
6-[5-(3-Methylphenyl)-1H-[1,2,3]triazol-4-yl]-[1,2,4]triazolo[1,5-a]pyridine;
6-[5-(4-Chlorophenyl)-1H-[1,2,3]triazol-4-yl]-[1,2,4]triazolo[1,5-a]pyridine;
6-[5-(4-Fluorophenyl)1H-[1,2,3]triazol-4-yl]-[1,2,4]triazolo[1,5-a]pyridine;
6-[5(4-Methylphenyl)-1H-[1,2,3]triazol-4-yl]-[1,2,4]triazolo[1,5-a]pyridine;
6-[5(3,4-Difluorophenyl)-1H-[1,2,3]triazol-4-yl]-[1,2,4]triazolo[1,5-a]pyridine;
6-[5-(2-Chlorophenyl)1H-[1,2,3]triazol-4-yl]-[1,2,4]triazolo[1,5-a]pyridine;
6-[5-(3-Chlorophenyl)-1H-[1,2,3]-triazol-4-yl]-4H-benzo[1,4]oxazin-3-one;
5-[5-(3-Chlorophenyl)-2H-[1,2,3]-triazol-4-yl]-benzo[1,2,5]thiadiazole;
5-[5-(3-Fluorophenyl)-2H-[1,2,3]-triazol-4-yl]-benzo[1,2,5]thiadiazole;
5-[5-(3-Bromophenyl)-2H-[1,2,3]-triazol-4-yl]-benzo[1,2,5]thiadiazole;
4-(3-Chlorophenyl)-5-(methoxyphenyl)-2H-[1,2,3]triazole;
4-(3-Fluorophenyl)-5-(4-methoxyphenyl)-2H-[1,2,3]triazole;
4-(3-Chlorophenyl)-5-(3-fluoro-4-methoxyphenyl)-2H-[1,2,3]triazole;
4-(3-Fluorophenyl)-5-(3-fluoro-4-methoxyphenyl)-2H-[1,2,3]triazole;
6-[5-(3-Chlorophenyl)-1H-[1,2,3]triazol-4-yl]-1-methyl-1H-benzoimidazole;
4-(3-Chlorophenyl)-5-(3-chloro-4-methoxyphenyl)-2H-[1,2,3]triazole; and
4-(3-Fluorophenyl)-5-(3-chloro-4-methoxyphenyl)-2H-[1,2,3]triazole.

Suitable pharmaceutically acceptable salts of the compounds of formula (I) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate and stearate.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably at least 10% of a compound of the formula (I) or pharmaceutically acceptable derivative thereof.

The term “C₁₋₆alkyl” as used herein whether on its own or as part of a larger group e.g. C₁₋₆alkoxy, means a straight or branched chain radical of 1 to 6 carbon atoms, unless the chain length is limited thereto, including, but not limited to methyl, ethyl, n-propyl, isopropyl, n-butyl, sec-butyl, isobutyl and tert-butyl.

C₁₋₆haloalkyl groups may contain one or more halo atoms, a particular C₁₋₆haloalkyl group that may be mentioned is CF₃.

The terms “halo” or “halogen” are used interchangeably herein to mean radicals derived from the elements chlorine, fluorine, iodine and bromine.

The term “cycloalkyl” as used herein means cyclic radicals, preferably of 3 to 7 carbons, including but not limited to cyclopropyl, cyclopentyl and cyclohexyl.

The term “ALK5 inhibitor” as used herein means a compound, other than inhibitory smads, e.g. smad6 and smad7, which selectively inhibits the ALK5 receptor preferentially over p38 or type II receptors.

The term “ALK5 mediated disease state” as used herein means any disease state which is mediated (or modulated) by ALK5, for example a disease which is modulated by the inhibition of the phosphorylation of smad ⅔ in the TGF-β1 signaling pathway.

The term “ulcers” as used herein includes but is not limited to, diabetic ulcers, chronic ulcers, gastric ulcers, and duodenal ulcers.

The compounds of formula (I) can be prepared by art-recognized procedures from known or commercially available starting materials. If the starting materials are unavailable from a commercial source, their synthesis is described herein, or they can be prepared by procedures known in the art.

Specifically, compounds of formula (I) may be prepared as illustrated in Scheme 1.

An aryl bromide (I) is coupled with trimethylsilylacetylene using a palladium catalyst in the presence of copper(I) iodide. The trimethylsilyl group is then removed under basic conditions with potaessium carbonate and the unmasked terminal acetylene (II) is coupled to a substituted bromobenzene (III) via palladium catalysis. The disubstituted acetylene (IV) is treated with trimethylsilylazide to afford a triazole (V) which may be alkylated with a suitable alkylating agent, L-R₃ where L is a leaving group, e.g. I, in the presence of potassium carbonate. The resulting isomers can be separated by chromatographic methods.

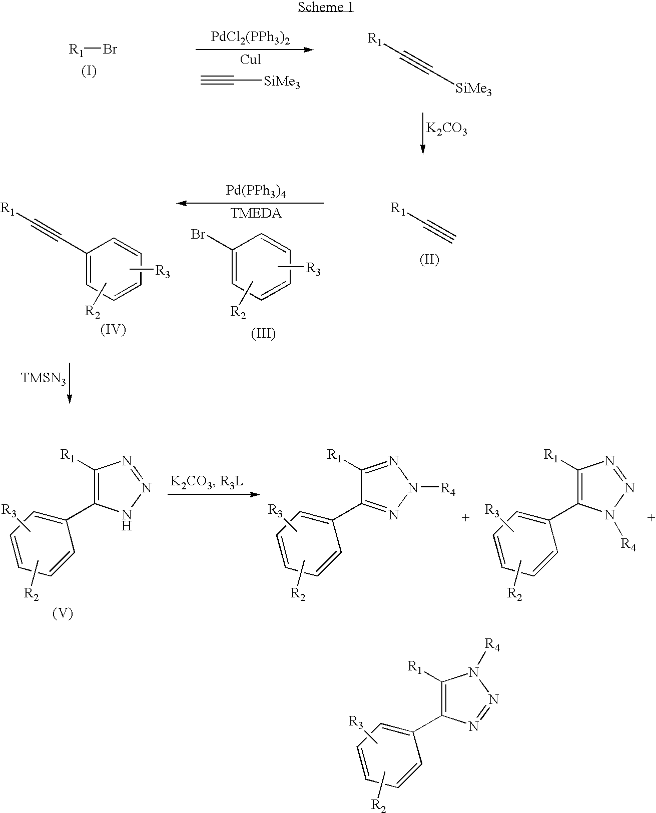

Further details for the preparation of compounds of formula (I) are found in the examples.

During the synthesis of the compounds of formula (I) labile functional groups in the intermediate compounds, e.g. hydroxy, carboxy and amino groups, may be protected. A comprehensive discussion of the ways in which various labile functional groups may be protected and methods for cleaving the resulting protected derivatives is given in for example Protective Groups in Organic Chemistry, T. W. Greene and P. G. M. Wuts, (Wiley-Interscience, New York, 2nd edition, 1991).

The compounds of formula (I) may be prepared singly or as compound libraries comprising at least 2, for example 5 to 1,000 compounds, and more preferably 10 to 100 compounds of formula (I). Libraries of compounds of formula (I) may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable salts thereof.

According to a further aspect of the present invention there is provided a method of treating a disease mediated by the ALK5 receptor in mammals, comprising administering to a mammal in need of such treatment, an effective amount of a compound of formula (I) or a pharmaceutically acceptable salt thereof.

According to a further aspect of the invention there is provided a compound of formula (I) or a pharmaceutically acceptable salt thereof, for use in therapy.

According to a further aspect of the present invention there is provided the use of a compound of formula (I) or a pharmaceutically acceptable salt thereof, in the manufacture of a medicament for the treatment of a disease mediated by the ALK5 receptor in mammals.

ALK5-mediated disease states, include, but are not limited to, chronic renal disease, acute renal disease, wound healing, arthritis, osteoporosis, kidney disease, congestive heart failure, ulcers, ocular disorders, corneal wounds, diabetic nephropathy, impaired neurological function, Alheimer's disease, atherosclerosis, peritoneal and sub-dermal abrasion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis.

By the term “treating” is meant either prophylactic or therapeutic therapy.

According to a further aspect of the present invention there is provided a method of inhibiting the TGF-β signaling pathway in mammals, for example, inhibiting the phosphorylation of smad2 or smad3 by the type I or activin-like kinase ALK5 receptor, which method comprises administering to a mammal in need of such treatment, an effective amount of a compound of formula (I) or a pharmaceutically acceptable salt thereof.

According to a further aspect of the present invention there is provided a method of inhibiting matrix formation in mammals by inhibiting the TGF-β signalling pathway, for example, inhibiting the phosphorylation of smad2 or smad3 by the type I or activin-like kinase ALK5 receptor, which method comprises administering to a mammal in need of such treatment, an effective amount of a compound of formula (I) or a pharmaceutically acceptable salt thereof.

The compounds of formula (I) and pharmaceutically acceptable salts thereof, may be administered in conventional dosage forms prepared by combining a compound of formula (I) with standard pharmaceutical carriers or diluents according to conventional procedures well known in the art. These procedures may involve mixing, granulating and compressing or dissolving the ingredients as appropriate to the desired preparation.

According to a further aspect of the present invention there is provided a pharmaceutical composition comprising a compound of formula (I), or a pharmaceutically acceptable salt thereof, and a pharmaceutically acceptable carrier or diluent.

The pharmaceutical compositions of the invention may be formulated for administration by any route, and include those in a form adapted for oral, topical or parenteral administration to mammals including humans.

The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognized by one of skill in the art that the optimal quantity and spacing of individual dosages of a formula (I) compound will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the formula (I) compound given per day for a defined number of days, can be ascertained by those skilled in the art using conventional course of treatment determination tests.

No toxicological effects are indicated when a compound of formula (I) or a pharmaceutically acceptable derivative thereof is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

The following examples are to be construed as merely illustrative and not a limitation on the scope of the invention in any way. In the Examples, mass spectra were performed using an Hitachi Perkin-Elmer RMU with chemical ionization technique (CI) or a Micromass Platform II instrument with electrospray (ES) ionization technique.

Abbreviations

CuI—copper iodide
DMF—dimethylformamide
EtOAc—ethyl acetate
MgSO₄—magnesium sulphate
NaHCO₃—sodium hydrogencarbonate
Na₂SO₄—sodium sulphate
Pd(PPh3)4—tetrakis(triphenylphosphine) palladium(0)
THF—tetrahydrofuran
TMEDA—tetramethylethylenediamine
   Preparation 1: N′-(5-Bromo-2-aminopyridine)-NN-dimethylformamidine

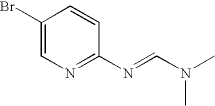

5-Bromo-2-aminopyridine (9.8 g, 56.6 mmol, 1 eq) was dissolved in dry DMF (20 ml) and dry dimethylformamide dimethylacetal (20 ml) under argon. The solution was refluxed at 130° C. for 16 h, allowed to cool, and the solvents removed. The resultant residue was used in the next stage without purification; m/z [APCIMS]: 228.0/230.0 [M+H]⁺.
Preparation 2: 6Bromo-[1,2,4]triazolo[1,5-a]pyridine

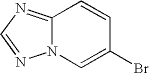

N′-(5-Bromo-2-aminopyridine)-N,N-dimethylformamidine (16.2 g, ˜56.6 mmol, 1 eq) was dissolved in methanol (90 ml) and pyridine (10 nm) under argon and cooled down to 0° C. To this was added, with stirring, hydroxylamine-O-sulfonic acid (7.3 g, 75.2 mmol, 1.3 eq) to form a purple suspension. This was allowed to reach room temperature and stirred for 16 h. After removing the solvents, the residue was suspended in aqueous sodium hydrogen carbonate (200 ml) and extracted with ethyl acetate (2×200 ml). The organic layer was then washed with water and brine (100 rut of each), dried (MgSO₄) and the solvent removed. Purification by flash chromatography on silica, eluting with a gradient solvent system of first 2:1 40-60° C. petroleum ether:ethyl acetate to 1:1 40-60° C. petroleum ether:ethyl acetate afforded the product as a pale yellow solid (5 g, 44.6%); ¹H NMR (250 MHz; CDCl₃) δ: 8.77 (1H, s), 8.34 (1H, s), 7.69 (1H, d), 7.65 (1H, d); m/z [APCIMS]: 198.0/200.0 [M+H]⁺.
Preparation 3: 6-Trimethylsilanylethynyl-[1,2,4]triazolo[1,5]pyridine

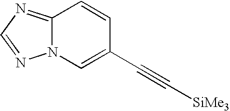

6-Bromo-[1,2,4]triazolo[1,5-a]pyridine (5 g, 25.26 mmol, 1 eq) was dissolved in THF (50 ml) and argon bubbled through the solution for 5 min. To this was added copper iodide (0.46 g, 2.53 mmol, 0.1 eq), dichlorobistriphenylphosphine (0.36 g, 0.51 mmol, 0.02 eq), and trimethylsilylacetylene (7.14 ml, 4.96 g, 50.52 mmol, 2 eq). Diisopropylamine (6.78 ml, 5.1 g, 50.52 mmol, 2 eq) was added dropwise to the solution and the resulting deep red suspension stirred under argon for 24 h. This was then filtered through celite, washing with an excess of ethyl acetate, and the solvents removed. The residue was then suspended in water (200 ml) and extracted with ethyl acetate (2×200 ml), and the organic layers combined, washed with water and brine (100 ml of each), dried (MgSO₄), and the solvent removed. Purification by flash chromatography over silica, eluting with 3:1 40-60° C. petroleum ether: ethyl acetate afforded the product as a pale yellow solid (2.9 g, 53.3%); ¹H NMR (400 MHz; CDCl₃) δ: 8.72 (1H, s), 8.36 (1H, s), 7.69 (1H, d), 7.54, (1H, d), 0.28 (9H, s); m/z [APCIMS]: 216 [M+H]⁺.
Preparation 4: 6-Ethynyl-11,2,4]triazolo[1,5-a]pyridine

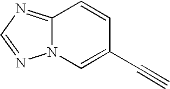

6-Trimethylsilanylethynyl-[1,2,4]triazolo[1,5-a]pyridine (2.9 g, 13.47 mmol, 1 eq) was dissolved in methanol and to this was added potassium carbonate (5.6 g, 40.4 mmol, 3 eq). The suspension was stirred for 2 h and the solvent removed. The residue was suspended in water (100 ml) and extracted with ethyl acetate (2×100 ml). The organic layers were then combined, washed with water and brine (50 ml of each), dried MgSO₄), and the solvent removed to give a pale orange solid (1.8 g, 95%) that was used in the next reaction without further purification; m/z [APCDIMS]: 144.1 [M+H]⁺.
Preparation 5: 6-(3-Chlorophenylethynyl)-[1,2,4]triazolo[1,5-a]pyridine

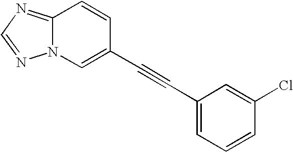

A stirred solution of 6-ethynyl-[1,2,4]triazolo[1,5-a]pyridine (693 mg, 4.846 mmol) in TMEDA (15 ml) and THF (15 ml) was degassed with argon. Pd(PPh₃)₄ (0.253 mg, 0.219 mmol, 0.05 eq), CuI (100 mg, 0.524 mmol, 0.1 eq) and 3-chloroiodobenzene (2.311 g, 9.69 mmol, 2 eq) were added, and the solution heated at 50° C. for 16 h under argon. The solvent was removed in vacuo and the residue partitioned between ethyl acetate (3×70 ml) and saturated aq. NaHCO₃ (70 ml). The ethyl acetate layers were combined, dried (Na₂SO₄), filtered and concentrated to dryness in vacuo. Silica gel chromatography, eluting with ethyl acetate/petroleum spirit (4:6), gave the product as a yellow solid (824 mg, 67%); CIMS: 254.1 [M+H]⁺.
Preparation 6: 5-Bromobenzo[1,2,5]thiadiazole

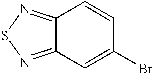

To 4-bromobenzene-1,2-diamine (17 g, 91 mmol) was added thionyl chloride (200 ml). One drop of DMF was added to the reaction mixture. The reaction mixture was heated at reflux under argon at 80° C. overnight. The reaction mixture was cooled to room temperature and added portionwise to ice in a large beaker and neutralised with solid sodium bicarbonate. The mixture was partitioned between ethyl acetate and water. The ethyl acetate layer was collected and dried (MgSO₄). The solvent was removed under reduced pressure. The title compound was isolated by column chromatography on silica gel eluting with 90% ethyl acetate/10% methanol. (12 g, 62%); ¹HNMR (250 MHz, CDCl₃) δ: 7.61 (1H, dd, J=9, 2 Hz), 7.82 (1H, d, J=9 Hz), 8.16 (1H, s).
Preparation 7: (4-Bromo-2-nitrophenoxy)acetic acid ethyl ester

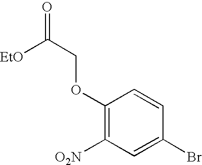

To a stirred solution of 4-bromo-2-nitrophenol (3.71 g, 17.0 mmol, 1.0 eq) in DMF (80 ml) at r.t. was added solid K₂CO₃ (4.70 g, 34.0 mmol, 2.0 eq). The mixture was heated at 40° C. for 3 h then allowed to cool to r.t. and partitioned between EtOAc and water. The aqueous phase was extracted with more EtOAc and the combined organic phase washed with water, brine and dried over MgSO₄. Concentration gave a yellow solid (5.01 g, 97%) which did not require further purification. ¹H NMR (250 MHz; CDCl₃) δ 8.00 (1H, d), 7.62 (1H, dd), 6.90 (1H, d), 4.76 (H, s), 4.26 (2H, q), 1.29 (3H, t).
Preparation 8: 6-Bromo-4H-benzo[1,4]oxazin-3-one

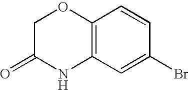

To a stirred solution of (4-bromo-2-nitrophenoxy)acetic acid ethyl ester (4.01 g, 13.2 mmol, 1.0 eq) in glacial acetic acid (70 ml) at r.t. was added iron powder (14.70 g, 264.0 mmol, 20.0 eq).

The mixture was stirred vigorously at 60° C. for 4 h then allowed to cool to r.t. The mixture was filtered through a pad of Kieselguhr, washing through with EtOAc, and the solution evaporated to dryness. The residue was partitioned between saturated aqueous NaHCO₃ solution and EtOAc. The aqueous was extracted with EtOAc and the combined organic phases washed with water, brine and dried over MgSO₄. Concentration gave the title compound (2.90 g, 97%) as a white solid which did not require purification. ¹H NMR (250 MHz; CDCl₃) δ: 10.79 (1H, br.s) 7.09-7.01 (2H, m), 6.91 (1H, d), 4.59 (2H, s).

### EXAMPLES

### Example 1

### 6-[5-(3-Chlorophenyl)-H-[1,2,3]triazol-4-yl]-[1,2,4]triazolo[1,5-a]pyridine

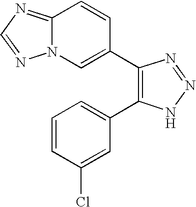

A stirred solution of 6-(3-chlorophenylethynyl)-[1,2,4]triazolo[1,5-a]pyridine (Preparation 5) (205 mg, 0.807 mmol) in DMF (1.1 ml) under argon was treated with azidotrimethylsilane (0.32 ml, 2.42 mmol), and heated at 130° C. for 19 h. The DMF was removed in vacuo and the mixture partitioned between ethyl acetate and brine. The ethyl acetate layer was dried (Na₂SO₄), filtered and concentrated to dryness in vacuo. The residue was purified by silica chromatography, eluting with petroleum spirit/ethyl acetate 2:1→neat ethyl acetate, giving an off-white solid (83 mg, 35%); ¹H NMR (250 MHz; CDCl₃) δ: 8.97 (1H, s), 8.42 (1H, s), 7.84 (1H, d), 7.74 (1H, dd), 7.62 (1H, d), 7.46 (3H, m); NH not observed; m/z [ESMS]: 297 [M+H]⁺.

### Example 2

### 6-[5-(3-Fluorophenyl)-1H-[1,2,3]triazol-4-yl]-[1,2,4]triazolo[1,5-a]pyridine

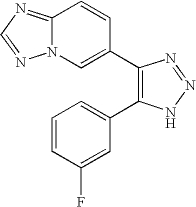

The title compound was prepared from 6-(3-fluorophenylethynyl)-[1,2,4]triazolo[1,5-a]pyridine (171 mg, 0.722 mmol) using a similar procedure to that described for Example 1. ¹H NMR (400 MHz; d₆-DMSO, free base) δ: 15.49 (NH, br.s), 9.03 (1H, s), 8.57 (1H, s), 7.93 (1H, d), 7.70 (1H, d), 7.48-7.27 (4H, m); m/z [ESMS]: 281 [M+H]⁺.

### Example 3

### 6-[5(3-Nitrophenyl 1H-[1,2,3]triazol-4-yl]-[1,2,4]triazolol[1,5-a]pyridine

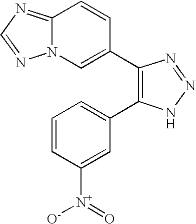

The title compound was prepared from 6-(3-nitrophenylethynyl)-[1,2,4]triazolo[1,5-a]pyridine (213 mg, 0.807 mmol) using a similar procedure to that described for Example 1. ¹H NMR (400 MHz; d₆-DMSO, free base) δ: 15.74 (NH, br.s), 9.15 (1H, s), 8.59 (1H, s), 8.40 (1H, s), 8.27 (1H, d), 7.95 (2H, br.s), 7.73 (2H, br.s); m/z [ESMS]: 308 [M+H]⁺.

### Example 4

### 6-[5-(3-Methylphenyl)-1H-[1,2,3]triazol-4-yl]-[1,2,4]triazolo[1,5-a]pyridine

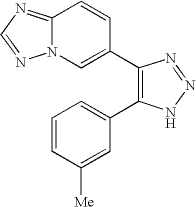

The title compound was prepared from 6-(3-methylphenylethynyl)-[1,2,4]triazolo[1,5-a]pyridine (188 mg, 0.805 mmol) using a similar procedure to that described for Example 1. ¹H NMR (250 MHz, CDCl₃, free base) δ: 9.16(1H, s), 8.43 (1H, s), 7.79 (2H, d), 741-7.28 (4H, m), 2.38 (3H, s); NH not observed; m/z [ESMS]: 277 [M+H]⁺.

### Example 5

### 6-[5-(4-Chlorophenyl)-1H-[1,2,3]triazol-4-yl]-[1,2,4]triazolo[1,5-a]pyridine

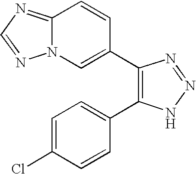

The title compound was prepared from 6-(4-chlorophenylethynyl)-[1,2,4]triazolo[1,5-a]pyridine (102 mg, 0.40 mmol) using a similar procedure to that described for Example 1. ¹H NMR (250 MHz; CD₃OD, free base) δ: 8.83 (1H, s), 8.35 (1H, s), 7.73 (1H, d), 7.69 (1H, d), 7.45 (2H, d), 7.36 (2H, d), NH not observed; [ESMS]: 297 [M+H]⁺.

### Example 6

### 6-[5(4-Fluorophenyl)-1H-[1,2,3]triazol-4-yl]-[1,2,4]triazolo[1,5-a]pyridine

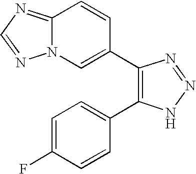

The title compound was prepared from 6-(3,4-fluorophenylethynyl)-[1,2,4]triazolo[1,5-a]pyridine (195 mg, 0.821 mmol) using a similar procedure to that described for Example 1. ¹H NMR (400 MHz; CDCl₃, free base) δ: 13.45 (NH, br.s), 9.12 (1H, s), 8.44 (1H, s), 7.83 (1H, d), 7.74 (1H, d), 7.57-7.51 (2H, m) 7.17-7.10 (2H, m); m/z [ESMS]: 281 [M+H]⁺.

### Example 7

### 6-[5(4-Methylphenyl)-1H-[1,2,3]triazol-4-yl]-[1,2,4]triazolo[1,5-a]pyridine

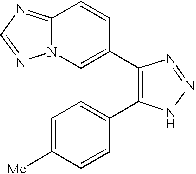

The title compound was prepared from 6-(4-methylphenylethynyl)-[1,2,4]triazolo[1,5-a]pyridine (180 mg, 0.773 mmol) using a similar procedure to that described for Example 1. ¹H NMR (400 MHz; DMSO, free base) δ: 15.33 (NH, br.s), 8.97 (1H, s), 8.54 (1H, s), 7.91 (1H, d), 7.70 (1H, d), 7.44 (2H, d) 7.27 (2H, br. s), 2.45 (3H, s); m/z [ESMS]: 277 [M+H]⁺.

### Example 8

### 6-[5-(3,4-Difluorophenyl)-1H-[1,2,3]triazol-4-yl]-[1,2,4]triazolo[1,5-a]pyridine

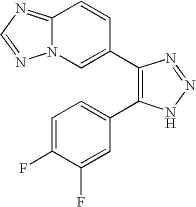

The title compound was prepared from 6-(3,4-difluorophenylethynyl)-[1,2,4]triazolo[1,5-a]pyridine (139 mg, 0.545 mmol) using a similar procedure to that described for Example 1. ¹H NMR (250 MHz; CDCl₃, free base) δ: 9.01 (1H, s), 8.43 (1H, s), 7.83 (1H, d), 7.70 (1H, d), 7.49-7.40 (1H, m), 7.19-7.30 (2H,m), NH not observed; m/z [ESMS]: 299 [M+H]⁺.

### Example 9

### 6-[5-(2-Chlorophenyl)-1H-[1,2,3]triazol-4-yl]-[1,2,4]triazolo[1,5]pyridine

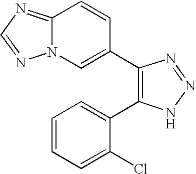

The title compound was prepared from 6-(2-chlorophenylethynyl)-[1,2,4]triazolo[1,5-a]pyridine (189 mg, 0.746 mmol) using a similar procedure to that described for Example 1. ¹H NMR (400 MFz; d₆-DMSO, free base) δ: 15.63 (1H, br.s), 8.74 (1H, s), 8.51 (1H, s), 7.91 (1H, d), 7.68-7.52 (5H, m), m/z[ESMS]: 297 [M+H]⁺.

### Example 10

### 6-[5-(3-Chlorophenyl)-1-[1,2,3]triazol-4-yl]-4H-benzo[1,4]oxazin-3-one

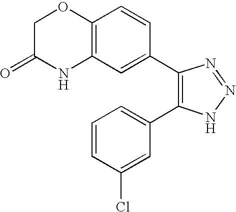

To a stirred suspension of 6-(3-chlorophenylethynyl)-4H-benzo[1,4]oxazin-3-one (0.311 g, 1.15 mmol, 1.0 eq) in dry DMF (1.5 ml) was added (trimethylsilyl)azide (0.397 g, 3.45 mmol, 3.0 eq). The mixture was degassed with argon for 5 mins and then heated in a sealed tube at 100° C. for a total of 72 h. More (trimethylsilyl)azide (0.397 g, 3.45 mmol, 3.0 eq) was added after 24 h. The mixture was allowed to cool then partitioned between water and EtOAc. The aqueous layer was extracted with more EtOAc and the combined organic phase washed with water, brine and dried over MgSO₄. Concentration gave a solid which was purified by flash column chromatography over silica, eluting with 50% EtOAc-petrol—EtOAc. The product was obtained as a yellow solid (0.063 g, 17%). ¹H NMR (250 MHz; DMSO-d⁶) (NMR very broad at r.t) δ: 10.80 (1H, br.s), 7.55-7.35 (4H, m), 7.20-6.90 (3H, m), 4.63 (2H, s), triazole N not observed; m/z [ESMS]: 327.2 [M+H]⁺.

### Example 11

### 5-[5-(3-Chlorophenyl-2H-[1,2,3]-triazol-4-yl]-benzo[1,2,5]thiadiazole

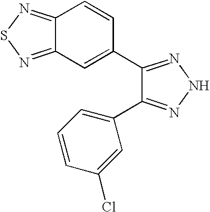

Prepared from 5-(3-chlorophenylethynyl)-benzo[1,2,5]thiadiazole using a similar procedure to that described for Example 10. ¹H NMR (400 MHz, CDCl₃) δ: 8.22 (1H, s), 8.01 (1H, d), 7.80 (1H, d), 7.37 (3H, m), 7.17 (1H, t), NH not observed.

### Example 12

### 5-[5-(3-Fluorophenyl-2H-[1,2,3]-triazol-4-yl]-benzo[1,2,5]thiadiazole

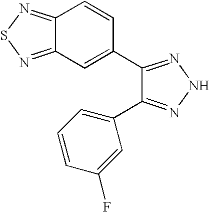

Prepared from 5-(3-fluorophenylethynyl)-benzo[1,2,5]thiadiazole using a similar procedure to that described for Example 10. ¹H NMR (400 MHz, CDCl₃): 8.24 (1H, s), 8.04 (1H, d), 7.84 (1H, d), 7.35 (3H, m), 7.15 (1H, t), NH not observed.

### Example 13

### 5-[5-(3-Bromophenyl-2H-[1,2,3]-triazol-4-yl]-benzo[1,2,5]thiadiazole

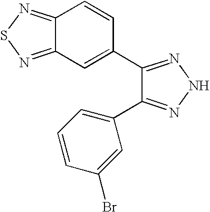

Prepared from 5-(3-bromophenylethynyl)-benzo[1,2,5]thiadiazole using a similar procedure to that described for Example 10. ¹H NMR (400 MHz, CDCl₃): 8.24 (1H, s), 8.02 (1H, d), 7.80 (1H, s), 7.56 (1H, d), 7.45 (1H, d), 7.26 (1H, t), NH not observed; m/z [APCIMS]: 358/360 [M+H⁺].

### Example 14

### 4-(3-Chlorophenyl (4-methoxyphenyl)-21-[1,2,3]triazole

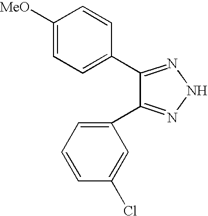

Prepared from 3-(4-methoxyphenylethynyl)chlorobenzene using a procedure similar to that described for Example 10. ¹H NMR (400 MHz, CDCl₃): 7.61 (1H, m), 7.46 (3H, m), 7.30 (2H, m), 6.93 (2H, m), 3.84 (3H, s), NH not observed; m/z [APCIMS]: 286.2 [M+H⁺].

### Example 15

### 4-(3-Fluorophenyl)-(4-methoxyphenyl)-2H-[1,2,3]triazole

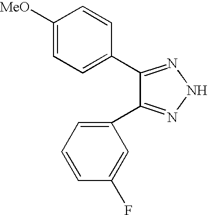

Prepared from 3-(4-methoxyphenylethynyl)fluorobenzene using a procedure similar to that described for Example 10. ¹H NMR (250 MHz, CDCl₃): 7.47 (2H, d), 7.35 (3H, m), 6.95 (1H, m), 6.92 (2H, d), 3.85 (3H, s), NH not observed; m/z [APCIMS]: 270.2 [M+H⁺].

### Example 16

### 4-(3-Chlorophenyl)-5-(3-fluoro-4-methoxyphenyl)-2H-[1,2,3]triazole

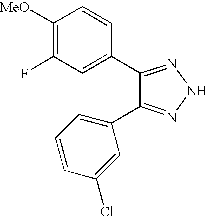

Prepared from 3-(3-fluoro-4-methoxyphenylethynyl)fluorobenzene using a procedure similar to that described for Example 10. ¹H NMR (400 MHz, CDCl₃) δ: 7.60 (1H, s), 7.37 (5H, m), 6.97 (1H, t), 3.92 (3H, s), NH not observed; m/z [APCIMS]: 304.1 [M+H⁺].

### Example 17

### 4-(3-Fluorophenyl)5-(3-fluoro-4-methoxyphenyl)-2H-[1,2,3]triazole

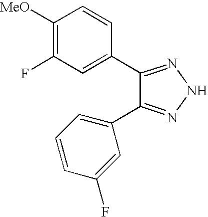

Prepared from 3-(3-fluoro-4-methoxyphenylethynyl)-fluorobenzene using a procedure similar to that described for Example 10. ¹H NMR (400 MHz, CDCl₃) δ: 7.33 (5H, m), 7.09 (1H, m), 6.97 (1H, t), 3.93 (3H, s), NH not observed; m/z [APCIMS]: 288.2 [M+H⁺].

### Example 18

### 6-[5-(3-Chlorophenyl)1H-[1,2,3]triazol-4-yl]-1-methyl-1H-benzoimidazole

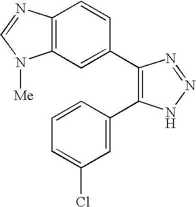

Prepared from 6-(3-chlorophenylethynyl)-1-methyl-1H-benzimidazole using a procedure similar to that described for Example 10. ¹H NMR (HCl salt, 400 MHz, MeOH) δ: 9.45 (1H, s), 8.13 (1H, s), 7.89 (1H, d), 7.76 (1H, d), 7.55 (1H, s), 7.447.37 (3H, m), 4.12 (3H, s), NH not observed.); m/z [APCIMS]: 267 [M+H⁺].

### Example 19

### 4-(3-Chlorophenyl)-5-(3-chloro-4-methoxyphenyl)-2H-[1,2,3]triazole

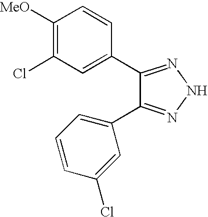

Prepared from 33-chloro-4-methoxyphenylethynyl)chlorobenzene using a procedure similar to that described for Example 10. ¹H NMR (400 MHz, CDCl₃) δ: 7.60 (1H, d), 7.50 (1H, d), 7.34 (4H, m), 6.93 (1H, t), 3.92 (3H, s), NH not observed.

### Example 20

### 4-(3-Fluorophenyl)-5-(3-chloro-4-methoxyphenyl)-2H-[1,2,3]triazole

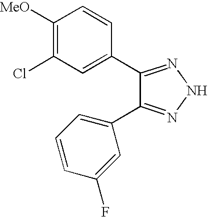

Prepared from 3-(3-chloro-4-methoxyphenylethynyl)fluorobenzene (500 mg, 1.92.mmol, 1 eq) using a procedure similar to that described for Example 10. ¹H NMR (400 MHz, CDCl₃) δ: 7.57 (1H, d), 7.46 (1H, dd), 7.25 (3H, m), 7.1 (1H, t), 6.94(1H, d), 3.93 (3H, s), NH not observed.

Biological Data

The biological activity of the compounds of the invention may be assessed using the following assays:

Method for Evaluating ALK5 Kinase Phosphorylation of smad3

Basic Flash-Plates (NEN Life Sciences) were coated by pipetting 100 micro liter of 0.1 molar sodium bicarbonate (pH 7.6), containing 150 nanograms of the fusion protein glutathion-S-transferase-smad3/100 micro liter of coating buffer. Plates were covered and incubated at room temperature for 10-24 hours. Then the plates were washed 2 times with 200 micro liter of coating buffer (0.1 molar sodium bicarbonate) and allowed to air dry for 2-4 hours.

For the phosphorylation reaction each well received 90 microliter containing 50 millimolar HEPES buffer (pH 7.4); 5 millimolar MgCl₂; 1 millimolar CaCl₂; 1 millimolar dithiothreitol; 100 micromolar guanosine triphosphate; 0.5 micro Ci/well gamma³³P-adenosine triphosphate (NEN Life Sciences) and 400 nanograms of a fusion protein of glutathion-S-transferase at the N-terminal end of the kinase domain of ALK5 (GST-ALK5). Background counts were measured by not adding any GST-ALK5. Inhibitors of ALK5 were evaluated by determining the activity of the enzyme in the presence of various compounds. Plates were incubated for 3 hours at 30° C. After incubation the assay buffer was removed by aspiration and the wells were washed 3 times with 200 microliter cold 10 millimolar sodium pyrophosphate in phosphate buffered saline. The last wash was aspirated and blotted plate dry. Plate was then counted on a Packard TopCount.

Fluorescence Anisotropy Kinase Binding Assay

The kinase enzyme, fluorescent ligand and a variable concentration of test compound are incubated together to reach thermodynamic equilibrium under conditions such that in the absence of test compound the fluorescent ligand is significantly (>50%) enzyme bound and in the presence of a sufficient concentration (>10×Kᵢ) of a potent inhibitor the anisotropy of the unbound fluorescent ligand is measurably different from the bound value.

The concentration of kinase enzyme should preferably be ≧1×K_{f}. The concentration of fluorescent ligand required will depend on the instrumentation used, and the fluorescent and physicochemical properties. The concentration used must be lower than the concentration of kinase enzyme, and preferably less than half the kinase enzyme concentration. A typical protocol is:

All components dissolved in Buffer of final composition 50 mM HEPES, pH 7.5, 1 mM CHAPS, 1 mM DTT, 10 mM MgCl₂ 2.5% DMSO.
ALK5 Enzyme concentration: 4 nM
Fluorescent ligand concentration: 1 nM
Test compound concentration: 0.1 nM-100 uM
Components incubated in 10 ul final volume in LJL HE 384 type B black microtitre plate until equilibrium reached (5-30 mins)
Fluorescence anisotropy read in LJL Acquest.
   Definitions:
Kᵢ=dissociation constant for inhibitor binding
K_{f}=dissociation constant for fluorescent ligand binding

The fluorescent ligand is the following compound: 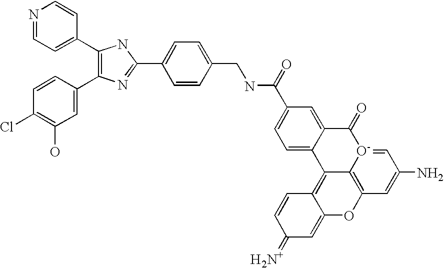
which is derived from 5-[2(4-aminomethylphenyl)-5-pyridin-4-yl-1H-imidazol-4-yl]-2-chlorophenol and rhodamine green.
Inhibition of Matrix Markers: Northern Blot Protocol

Data confirming activity in the enzyme assay was obtained as follows: A498 renal epithelial carcinoma cell lines were obtained from ATCC and grown in EMEM medium supplemented with 10% fetal calf serum, penicillin (5 units/ml) and streptomycin (5 ng/ml). A498 cells were grown to near confluence in 100 mm dishes, serum-starved for 24 hours, pretreated with compounds for 4 hours followed by a 10 ng/ml addition of TGF-beta1 (&D Systems, Inc., Minneapolis Minn.). Cells were exposed to TGF-beta1 for 24 hours. Cellular RNA was extracted by acid phenol/chloroform extraction (Chomczynski and Sacchi, 1987). Ten micrograms of total RNA were resolved by agarose gel electrophoresis and transferred to nylon membrane (GeneScreen, NEN Life Sciences, Boston Mass.). Membranes were probed with ³²P-labeled cDNA probes (Stratagene, La Jolla, Calif.) for fibronectin mRNA. Membranes were exposed to phosphorimaging plates and bands were visualized and quantified with ImageQuant software (Molecular Dynamics, Sunnyvale, Calif.).

Inhibition of Matrix Markers: Western Blot Protocol

Data confirming activity in the enzyme assay was obtained as follows:

Cells were grown to near confluence in flasks, starved overnight and treated with TGF-beta and compounds. Cells were washed at 24 or 48 hours after treatment with ice cold phosphate buffered saline, then 500 microliter of 2× loading buffer was added to plate and cells were scraped and collected in microcentrifuge tube. (2× loading buffer: 100 mM Tris-Cl, pH6.8, 4% sodium dodecyl sulfate, 0.2% bromophenol blue, 20% glycerol, 5% beta-mercapto-ethanol). Cells were lysed in tube and vortexed. Sample was boiled for 10 minutes. 20 microliters of sample was loaded on 7.5% polyacrylamide gel (BiORad) and electrophoresed.

Size fractionated proteins in gel were transferred to nitrocellulose membrane by semidry blotting. Membrane was blocked overnight with 5% powdered milk in phosphate buffer saline (PBS) and 0.05% Tween-20 at 4 degrees C. After 3 washes with PBS/Tween membranes were incubated with primary antibody for 4 hours at room temperature. After three washes with PBS/Tween membrane was incubated with secondary antibody for 1 hour at room temperature. Finally, a signal was visualized with ECL detection kit from Amersharm.

The compounds of this invention generally show ALK5 receptor modulator activity having IC₅₀ values in the range of 0.0001 to 10 μM.

1. A compound of formula (I), or a pharmaceutically acceptable salt thereof: 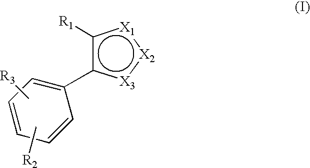
wherein R₁ is naphthyl or phenyl optionally substituted with one or more substituents selected from halo, —O—C₁₋₆alkyl, —S—C₁₋₆alkyl, C₁₋₆alkyl, C₁₋₆haloalkyl, —O—(CH₂)ₙ-Ph, —S—(CH₂)ₙ-Ph, cyano, phenyl, and CO₂R, wherein R is hydrogen or C₁₋₆alkyl, and n is 0, 1, 2 or 3; or R₁ is phenyl or pyridyl fused with an aromatic or non-aromatic cyclic ring of 5-7 members wherein said cyclic ring optionally contains up to three heteroatoms, independently selected from N, O and S, and N may be further optionally substituted by C₁₋₆ alkyl, and wherein the cyclic ring may be optionally substituted by ═O;
R₂ and R₃ are independently selected from H, C₁₋₆alkyl, C₁₋₆alkoxy, phenyl, NH(CH₂)ₙ-Ph, NH—C₁₋₆alkyl, halo, alkoxy, CN, NO₂, CONHR and SO₂NHR;
two of X₁, X₂ and X₃ are N and the other is NR₄ wherein R₄ is hydrogen, C₁₋₆alkyl, C₃₋₇cycloalkyl, —(CH₂)ₚ—CN, —(CH₂)ₚ—CO₂H, —(CH₂)ₚ—CONHR₅R₆, —(CH₂)ₚCOR₅, —(CH₂)_{q}(OR₇)₂, (CH₂)ₚOR₅, —(CH₂)_{q}—CH═CH—CN, (CH₂)_{q}—CH═CH—CO₂H, —(CH₂)ₚ—CH═CH—CONHR₅R₆, —(CH₂)ₚNHCOR₈ or —(CH₂)ₚNR₉R₁₀;
R₅ and R₆ are independently hydrogen or C₁₋₄alkyl;
R₇ is C₁₋₆alkyl;
R₈ is C₁₋₇alkyl, or optionally substituted aryl, heteroaryl, arylC₁₋₆alkyl or heteroaryl C₁₋₆alkyl;
R₉ and R₁₀ are independently selected from hydrogen, C₁₋₆alkyl, aryl and arylC₁₋₆alkyl;
p is 0-4; and
q is 1-4.

2. A compound of according to claim 1 wherein R¹ is phenyl optionally substituted by halo, or R¹ is phenyl or pyridyl fused with a 5- to 7-membered aromatic or non-aromatic ring wherein said ring optionally contains up to three heteroatoms, independently selected from N, O and S, and N may be further optionally substituted by C₁₋₆ alkyl, and wherein the cyclic ring may be optionally substituted by ═O.

3. A compound according to claim 2 wherein R₁ represents 4-methoxyphenyl, 3-fluoro-4-methoxyphenyl, 3-chlorophenyl, 3-fluoro-4-methoxyphenyl or 3-chloro-4-methoxyphenyl, or R₁ represents benzo[1,2,5]thiadiazolyl, [1,2,4]triazolo[1,5-a]pyridyl, dihydrobenzofuranyl, 2,3-dihydrobenzo[1,4]dioxinyl, benzimidazolyl, C₁₋₆alkylbenzimidazolyl, benzo[1,4]oxazinyl-3-one or benzo[1,4]oxazinyl.

4. A compound according to claim 1 wherein R₂ is positioned meta to the point of attachment to the triazole.

5. A compound according to claim 1 wherein R₂ is halo, C₁₋₆ alkyl or NO₂.

6. A compound according to formula (I) selected from:
6-[5-(3-Chlorophenyl)-1H-[1,2,3]-triazol-4-yl]-[1,2,4]triazolo[1,5-a]pyridine;
6-[5-(3-Fluorophenyl)-1H-[1,2,3]triazol-4-yl]-[1,2,4]-triazolo[1,5-a]pyridine;
6-[5-(3-Nitrophenyl)-1H-[1,2,3]triazol-4-yl]-[1,2,4]triazolo[1,5-a]pyridine;
6-[5-(3-Methylphenyl)-1H-[1,2,3]triazol-4-yl]-[1,2,4]triazolo[1,5-a]pyridine;
6-[5-(4-Chlorophenyl)-1H-[1,2,3]triazol-4-yl]-[1,2,4]triazolo[1,5-a]pyridine;
6-[5-(4-Fluorophenyl)-1H-[1,2,3]triazol-4-yl]-[1,2,4]triazolo[1,5-a]pyridine;
6-[5-(4-Methylphenyl)-1H-[1,2,3]triazol-4-yl]-[1,2,4]-triazolo[1,5-a]pyridine;
6-[5-(3,4-Difluorophenyl)-1H-[1,2,3]-triazol-4-yl]-[1,2,4]triazolo[1,5-a]pyridine;
6-[5-(2-Chlorophenyl)-1H-[1,2,3]triazol-4-yl]-[1,2,4]triazolo[1,5-a]pyridine;
6-[5-(3-Chlorophenyl)-1H-[1,2,3]triazol-4-yl]-4H-benzo[1,4]oxazin-3-one;
5-[5-(3-Chlorophenyl-2H-[1,2,3]-triazol-4-yl]-benzo[1,2,5]thiadiazole;
5-[5-(3-Fluorophenyl-2H-[1,2,3]-triazol-4-yl]-benzo[1,2,5]thiadiazole;
5-[5-(3-Bromophenyl-2H-[1,2,3]-triazol-4-yl]-benzo[1,2,5]thiadiazole;
4-(3-Chlorophenyl)-5-(4-methoxyphenyl)-2H-[1,2,3]triazole;
4-(3-Fluorophenyl)-5-(4-methoxyphenyl)-2H-[1,2,3]triazole;
4-(3-Chlorophenyl)-5-(3-fluoro-4-methoxyphenyl)-2H-[1,2,3]triazole;
4-(3-Fluorophenyl)-5-(3-fluoro-4-methoxyphenyl)-2H-[1,2,3]triazole;
6-[5-(3-Chlorophenyl)-1H-[1,2,3]triazol-4-yl]-1-methyl-1H-benzoimidazole;
4-(3-Chlorophenyl)-5-(3-chloro-4-methoxyphenyl)-2H-[1,2,3]triazole; and
4-(3-Fluorophenyl)-5-(3-chloro-4-methoxyphenyl)-2H-[1,2,3]triazole
or a pharmaceutically acceptable salt thereof.

7. A pharmaceutical composition comprising a compound according to claim 1, or a pharmaceutically acceptable salt thereof, and a pharmaceutically acceptable carrier or diluent.

8-9. Cancelled.

10. A method of inhibiting the TGF-β signaling pathway in mammals, comprising administering to a mammal, comprising administering to a mammal in need of such treatment, a therapeutically effective amount of a compound according to claim 1, or a pharmaceutically acceptable salt thereof.

11. A method for treating a disease selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis, and restenosis, comprising administering to a mammal in need of such treatment, a therapeutically effective amount of a compound according claim 1 or a pharmaceutically acceptable salt thereof.

12. A method for inhibiting matrix formation in mammals, comprising administering to a mammal, a therapeutically effective amount of a compound according to claim 1 or a pharmaceutically acceptable salt thereof.
